(19) United States
(12) Patent Application Publication
(10) Pub. No.: US 2022/0016559 A1
(43) Pub. Date: Jan. 20, 2022

(54) "Healthy Home Air Purification System"

(71) Applicant: Steven Michael Solseth, Oceanside, CA (US)

(72) Inventor: Steven Michael Solseth, Oceanside, CA (US)

(21) Appl. No.: 16/932,535

(22) Filed: Jul. 17, 2020

(51) Int. Cl. B01D 46/00; A61L 9/20

(52) U.S. Cl. CPC B01D 46/0028; B01D 46/0005; A61L 2209/14; B01D 2279/65; A61L 2209/12; A61L 9/20

(57) ABSTRACT

One embodiment of the purification system is the Base Support Frame A's ability to support a properly centered UVC light. Another embodiment is the Filter Frame B's ability to strengthen and hold in place a HEPA filter. In addition, locating the unit in the HVAC return ensures optimal air purification opportunities.

### PRIORITY CLAIM

Provisional Patent App. Ser. No. 63/101,203 filed 2020 Apr. 21

### FIELD OF THE INVENTION

This invention relates to the purification of air by combining the benefits of utilizing both a High-Efficiency Particulate Air (hereinafter “HEPA”) filter and an Ultraviolet germicidal bulbs (hereinafter “UVC light”), capable of trapping and penetrating unhealthy air particulates and eliminating airborne novel Coronavirus exposed to UVC light.

A Base Frame (“A”) is designed to hold a centered UVC light and power cord installed in a residential Heating, Venting and Air Conditioning (hereinafter “HVAC”) Return. A separate Frame (“B”) designed to accept a HEPA filter, is securely held in place and attached to the Base Frame A with two (2) metal hinges.

This invention relates to the purification of air in a residence by using scientifically proven methods used in hospital rooms, clean rooms, etc. Properly installed, the unit should eliminate 99% of allergens, bacteria, mold, spores, and pollen, while dramatically reducing the risk of novel Coronavirus exposure.

### BACKGROUND OF THE INVENTION

A new invention with no known prior art.

### PREAMBLE

The petitioner whose signature appears on the declaration attached respectfully requests that Letters Patent be granted to such petitioner for the new and original utility set forth in the following specification. A Patent Application Declaration, Field of Invention, Utility Patent Application—Preamble and Specification, Background Summary, Summary of Invention, Alternative Embodiments, Claims, Conclusion, Ramifications and Scope, and four (4) sheets of drawings are attached.

### SPECIFICATION

The undersigned has invented a new, original, and functional utility design entitled “Healthy Home Air Purification System” of which the following are specifications. Reference is made to the accompanying drawings which form a part hereof the figures of which are described as follows:

### DRAWING FIGURES

FIG. 1 is the Base Support Frame

FIG. 2 is the Air Filter Frame

FIG. 3 is UVC Light and Fixture

FIG. 4 is the Assembled Unit

### CROSS-REFERENCE TO RELATED APPLICATIONS: None

### STATEMENT REGARDING FEDERALLY SPONSORED RESEARCH

None

### BACKGROUND SUMMARY

In a typical home the only form of air cleaning is one (1) average quality air filter located in the HVAC air handler. Most of these will not eliminate all the allergens, bacteria, mold, dust, pollens, spores and other unhealthy particulates in the air. More importantly none of these air filters will eliminate the novel Coronavirus. Operating rooms and ICU Covid19 wards are sterilized using a combination of HEPA filters and UVC lights. The Healthy Home Air Purification System was designed to utilize this hospital technology and apply it to homes in the hopes of achieving the same sterilized air by utilizing this type of technology.

By combining Frame A, designed to properly position a support for a UVC light, with a second Frame B, which holds a HEPA filter securely in place, this system duplicates the sterilizing technology. Locating the purification unit in the HVAC return ensures that all household air will pass through and be treated. While we cannot guarantee 100% totally virus-free, sanitized air, this system provides the most advanced technology currently available.

The addition of a HEPA filter that traps allergens, bacteria, dust, spores and pollen in the return will facilitate keeping the supply line between the return and the air hander uncontaminated.

The Far UVC light has proven successful in eliminating both the SARs and MERs viruses by destroying their ability to reproduce. The same results are being achieved in fighting the novel Coronavirus in laboratories and hospitals across the country.

The Far UVC light cannot cause damage by penetrating the outer layer of the skin or eyes and thus is a major advance over the UVA and UVB lights. Research has shown that the Far UVC light can eradicate airborne viruses without harming people.

### SUMMARY OF THE INVENTION

It is therefore an object of this invention to provide au innovative Home Air Purification System.

It is a further object of this invention to provide such an Air Purification System that utilizes the health benefits of a HEPA filter and
technology proven to eliminate the novel Coronavirus, allergens, bacteria, mold, dust, pollens, spores and other unhealthy particulates in the air by exposure to UVC light,
the Base Frame A which will accept the UVC light and power cord, is attached to the HVAC return with sheet metal screws, and
the HEPA Filter Support Frame B which is held in the proper position by two (2) metal hinges.
which does not require modification to any existing HVAC components other than the return installation,
which works on a continuous basis for as long as the HVAC unit is engaged,
which permits the maximum amount of air to flow with a minimum amount of blockage occurring,
which does not require excessive power,
which has no moving parts and requires very little maintenance,
which is easily installed in an existing HVAC return,
which has air purification available on demand at the thermostat control unit
Which adds HEPA benefits by inserting Filter Frame Unit in the return.

This invention features a method of purifying air and eliminating viruses in a home by installing the Frame units, with the UVC light and HEPA filter in place in the HVAC return.

This invention is a result of the realization that current technology of utilizing both a HEPA filter and the UVC light to sanitize an operating room could be applied to a home and give similar results. By designing two separate frames and connecting them to form one (1) unit, both the filter and UVC light will properly function together. Installation in the HVAC return ensures all household air will pass through the system continuously.

### ALTERNATIVE EMBODIMENTS

There are various possibilities regarding installing a UVC light inside an HVAC system, such as the air handler, in the air return, or additional external air filtration units with a UVC light commercially installed.

Advantages

From the preceding description some embodiments of the Healthy Home Purification System become evident: the air then exposed to the UVC light and then passing through a REM filter and becomes purified. The Air Purification System is designed to securely hold the centered position of the UVC light and properly align the Air Filter Frame B to the Base Support Frame A.

Conclusions

Accordingly, the reader will see embodiments of the Air Purification System will function as designed and are easily assembled. UVC lights and HEPA filters are available from numerous manufacturers and simple to install.

Furthermore, this Air Purification System utilizes the most advanced technology available today to sterilize air. Applying this technology to a home will provide healthy, virtually virus-free, pure, sanitized air.

### FIGURES

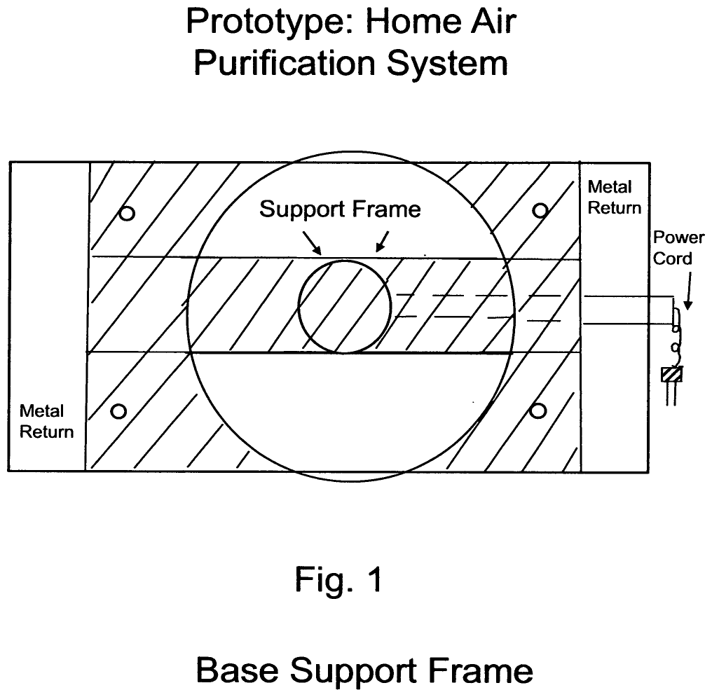

FIG. 1 (Front View)
The Base Support Frame A positions the centered UVC light on the support bar.
Cut-out areas allow air passage.
The Base Support Frame A is attached to the HVAC return with four (4) sheet metal screws.

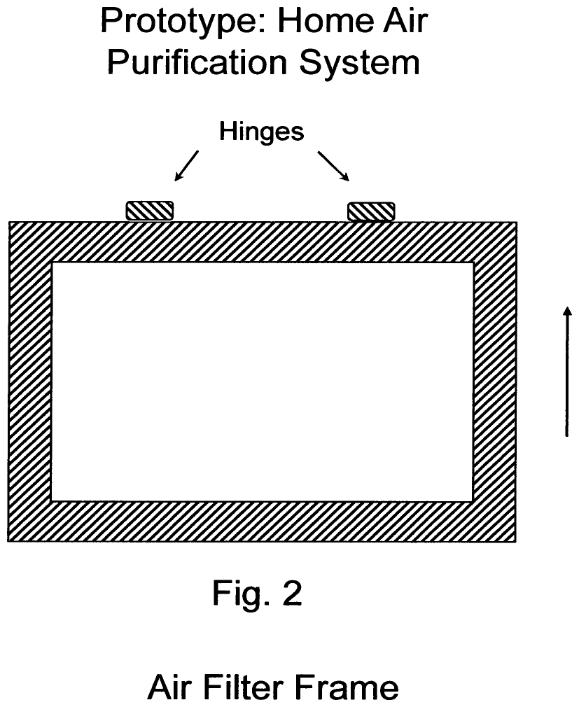

FIG. 2 (Front View)
Filter Frame B covers the side and edges of a HEPA filter.
Two (2) hinges attach HEPA Filter Frame B to top of Base Support Frame A. (Ref. FIG. 1)

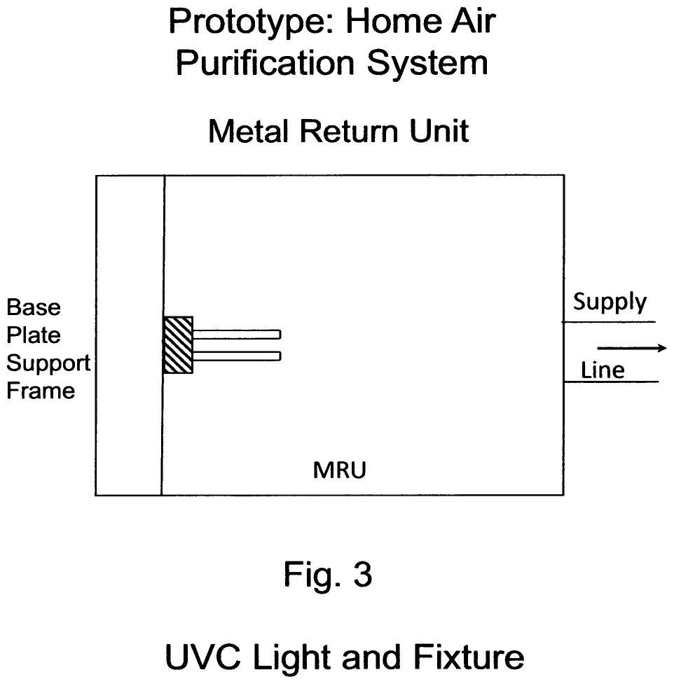

FIG. 3 (Side View)
The UVC light and fixture are positioned at the center of the Support Bar.
The UVC light fixture attaches to the Support Bar using three (3) screws.

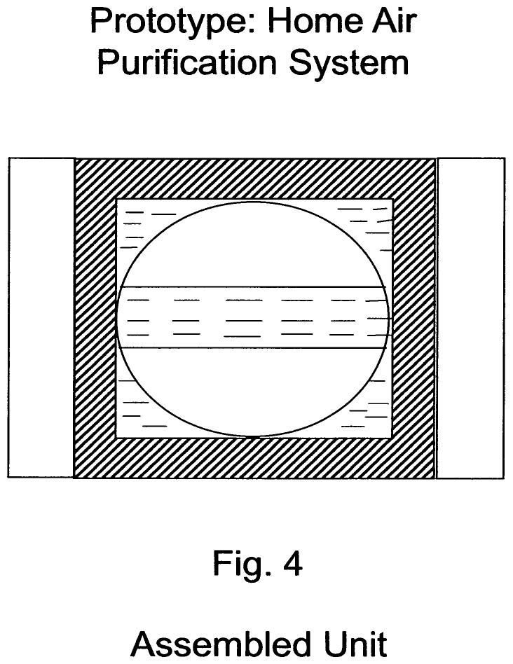

FIG. 4 (Front View)
Shows the Fully Assembled Unit.
No HEPA filter material is shown as filters are not a part of this patent.
The UVC light is not a part of this patent.

### CONCLUSION, RAMIFICATIONS AND SCOPE

The reader will see from the preceding description, several advantages of the embodiments of the Air Purification System become evident:
a. The use of UVC light to destroy the novel Coronavirus is the best technology currently available.
b. A centered UVC light position on a support beam optimizes UVC exposure of the passing air.
c. The cut-out sections of the Base Support Frame A allow powerful air passage.
d. The use of an Air Filter Frame B gives additional strength to the paper filter and to the final Assembled Unit when hinges are attached.
e. Frame B is designed to accept the insertion of a HEPA filter capable of trapping 99% of airborne particulates.
f. Encasing only the outer edges of a HEPA filter allows maximum air flow.
g. Installing the Purification System in the HVAC return will filter all the air in a home repeatedly.
h. Installing the Unit is a simple procedure using four (4) sheet metal screws.
i. A HEPA filter alone may trap the novel Coronavirus but will not destroy it.
j. When the novel Coronaviruses which are trapped in the HEPA filter are exposed to the UVC light they are destroyed.
k. Installing the Frame in the HVAC return to hold a HEPA filter adds to the Air Purification effectiveness.

What is claimed is:

1. A method of connecting two (2) Frames together in a manner capable of producing purified virus-free air in a home, the system consisting of: a) the Base Frame A that provides support for a UVC light and electric power cord, b) Frame B holding a HEPA filter in the correct position, and c) two (2) standard hinges connecting Frame A and Frame B. Base Frame A will accept and support the UVC light and electric power cord to destroy all viruses and bacteria exposed to the light; Frame B is capable of holding a HEPA filter in the correct position to filter all larger particles, including the 0.0125 micron novel Coronavirus particles; and two (2) standard 3 cm hinges joining Frame A and Frame B together complete the Assembled Unit.

2. The Air Purification System of claim 1 in which the Frames are made of a plastic composite material, metal, or a combination of these materials.

3. The Air Purification System of claim 1 in which the UVC light and the electric cord is attached to the Base Frame A, while allowing air to pass near the UVC light.

4. The Air Purification System of claim 1 in which the Base Frame A is fastened to the HVAC return with sheet metal screws.

5. The Air Purification System of claim 1 in which two (2) areas are cut-out to allow maximum air passage.

6. The Air Purification System of claim 1 in which Frame B encases all four (4) sides and edges of a HEPA filter, while allowing maximum air passage.

7. The Air Purification System of claim 1 in which two (2) 3 cm hinges are attached to the top of Frame A and B securing both as one Assembled Unit.

8. A method of incorporating a UVC light and a HEPA filter in two (2) separate Frames, which when attached together and installed in the HVAC return will purify the air of novel Coronaviruses, allergens, bacteria, mold, pollutants and other unhealthy air particulates.

9. A method of adding a HEPA filter to a HVAC return utilizing a Filter Frame.

10. A method where a UVC light is utilized to destroy viruses and bacteria trapped in the air filtration device or material.
